(19) United States
(12) Patent Application Publication
(10) Pub. No.: US 2008/0262578 A1
(43) Pub. Date: Oct. 23, 2008

(54) ELECTRODES USING TWO-PHASE HEAT TRANSFER AND MULTI-VARIATE ALGORITHMS FOR IMPROVED TEMPERATURE MONITORING AND CONTROL

(76) Inventors: Edwin Langberg, Sanford, FL (US); Julia Langberg, Sanford, FL (US); Jonathan Langberg, Atlanta, GA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/969,769

(22) Filed: Jan. 4, 2008

Related U.S. Application Data
(63) Continuation of application No. PCT/US2006/026189, filed on Jul. 5, 2006.
(60) Provisional application No. 60/696,697, filed on Jul. 5, 2005.

(51) Int. Cl. A61F 7/00

(52) U.S. Cl. 607/101

(57) ABSTRACT

A tissue hyperthermia system and method improves temperature monitoring and control along an energy emitter such as an RF electrode. A two-phase heat transfer system includes a material within an enclosed vessel that is thermally coupled to the electrode. Energizing the electrode to an operating condition emits energy into tissue and heats at least to a threshold temperature wherein the material undergoes a phase transformation within the vessel between a liquid phase and a vapor phase. The phase change assists in cooling, monitoring, and control of emitter temperature. Algorithms estimate maximum temperature either at the emitter or in tissue adjacent the emitter based on monitored parameters at the vessel. Multivariate algorithms use simultaneous power and temperature readings to estimate actual regional temperature, including electrode or tissue hot-spot temperature. A multivariate algorithm is based in particular upon time-dependent aspects of a pulsed RF operating mode. The multi-variate algorithms benefit temperature monitoring and control either together with the two-phase heat transfer system or with other more conventional devices.

### CROSS-REFERENCE TO RELATED APPLICATIONS

This application claims priority from, and is a 35 U.S.C. § 111 (a) continuation of, co-pending PCT international application serial number PCT/US2006/026189, filed on Jul. 5, 2006, incorporated herein by reference in its entirety, which claims priority to U.S. provisional application Ser. No. 60/696,697, filed on Jul. 5, 2005, incorporated herein by reference in its entirety.

This application is related to PCT Publication Nos. WO 2007/005963 A2 and WO 2007/005963 A3, each of which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

The present invention relates generally to medical devices. More specifically, the present invention relates to a radiofrequency (RF) ablation catheter system. Still more specifically, it relates to RF ablation catheter systems and methods for improved monitoring, control, and cooling of the ablation electrode.

2. Description of Related Art

Temperature measurement is critical in achieving success during RF catheter ablation of cardiac arrhythmias. The lesion size and shape are a function of the temperature of the ablated tissue: Tissue temperature must be high enough to sufficiently heat a desired volume of tissue to form a desired lesion. However, excessive heating of tissue may produce undesirable effects, including coagulum formation, charring, or perforation.

RF energy is supplied to an ablating electrode typically made of solid metal, such as for example platinum or stainless steel, and located at the tip of the catheter shaft. The temperature of the heated tissue is roughly estimated by monitoring the temperature at the ablating electrode. Such monitoring is typically performed by a thermistor or thermocouple temperature transducer attached at a location on the ablating electrode. Appropriate wiring that leads through the catheter shaft connects the ablating electrode to an RF generator; and, the temperature transducer is connected to a controller that receives a temperature-related signal. Both the RF generator and controller are located in a system console. The console provides an indication of RF power and catheter temperature, and allows manual or closed-loop adjustments of RF power output.

During RF ablation, heat flow and temperature of the electrode-tissue interface vary considerably over the surface of the ablating electrode. For example, one side of the electrode may be in firm contact with tissue while the other side of the electrode is cooled by blood flow. In spite of the relatively good thermal conductivity of the metallic electrode, significant temperature gradients may exist in the electrode. Animal studies have shown that the temperature transducer markedly underestimates the hottest tissue region, often by as much as 40° C.

Errors in temperature measurement are believed to be generally due to at least the following:

1. A hot spot on the electrode in an exemplary operating environment is typically at about 65° C. whereas a coolest region may be at about 40° C. The location of these two spots moves unpredictably on the electrode surface during operation. Temperature indication depends critically on the instantaneous distance of the location of the temperature transducer with respect to the electrode temperature extremes and this distance variability may introduce as much as 25° C. error.

2. By the typical nature of RF heating, the hottest tissue temperature is typically 0.5 mm-1 mm away from the electrode and therefore there is a significant temperature differential between the tissue hot spot and the electrode hot spot. The variable temperature difference between the electrode hot spot and the tissue hot spot may be in many instances about 15° C.

3. During ablation there is often dramatic variation in ablation electrode location, contact pressure and convective cooling. This can produce very rapid changes in local heating. The large thermal mass of the ablation electrode delays the measurement of these rapid fluctuations, increasing the risk of overheating at the hottest spot.

Limiting maximum RF power is sometimes used to reduce the risks associated with the problems described above. However, this generally will increase the probability of inadequate lesion size.

Some attempts to address the problem of electrode temperature measurement error have been previously disclosed and investigated. According to one example, an RF ablation catheter tip electrode is provided with multiple sensors. According to another example, a temperature transducer is located at the very distal end of the ablating electrode with a hopeful assumption that this is the location of the hot spot. According to still a further example, a catheter is provided with florescent temperature sensing on the interior surface of an electrode shell accomplished at the cost of substantial complexity.

The following US Patents are herein incorporated in their entirety by reference thereto: U.S. Pat. No. 5,688,266; U.S. Pat. No. 6,616,657; and U.S. Pat. No. 6,890,307 B2.

However, despite the previous attempts to provide an adequate solution, these prior approaches fall short of achieving optimally accurate measurements and thus control. A need still exists to optimize ablation procedures and to prevent tissue trauma due to overheating.

In particular, a need still exists for a system and method that provides real-time estimation and monitoring of tissue temperature or of RF tissue dosimetry, and in particular relation to “hottest” electrode and tissue temperature during operation. An ability for improved temperature monitoring that more accurately and reliably estimates the highest temperature reached on the electrode surface would be of tremendous value to the field. A need also exists to provide more detailed, more localized, and generally more useful information about the ablation thermal environment in order to accurately estimate tissue temperature.

### BRIEF SUMMARY OF THE INVENTION

One aspect of the invention is a system, and related method, that optimizes thermal tissue ablation procedures.

Another aspect of the invention is a system, and related method, that significantly limits or prevents undesirable tissue trauma and coagulum formation due to overheating during targeted thermal tissue ablation.

Another aspect of the invention is a system, and related method, that provides real-time estimation and monitoring of hottest tissue temperature or thermal dosimetry, and in particularly beneficial modes of RF tissue dosimetry.

Another aspect of the invention is a system, and related method, that provides improved temperature monitoring that reliably estimates the highest temperature reached on the electrode surface of electrical ablation, and in particularly beneficial modes of RF ablation.

Another aspect of the invention is a system, and related method that provides information about the ablation thermal environment during thermal tissue ablation that is useful for accurately estimating tissue temperature.

Another aspect of the invention is a novel system, and related method, that couples a two-phase heat transfer mechanism and process to thermal ablation energy emitters on ablation catheters or devices.

According to one mode, the two-phase heat transfer mechanism is coupled to at least one RF electrode.

According to another mode, an electrode temperature monitoring system, and related method, is coupled to the two-phase heat transfer mechanism and process.

According to another mode, a thermal regulator is provided that uses information associated with the two-phase heat transfer mechanism and process for regulating a thermal treatment apparatus such as an RF catheter for cardiac ablation.

In one highly beneficial embodiment of the various modes described, a vessel is provided within an interior space defined within an electrode. The vessel is enclosed by the electrode and a diaphragm, and is filled with a volume of coolant fluid. Upon energizing and heating the electrode, heat transfer through the interior of the electrode is based at least in part on a liquid-vapor phase transformation of the fluid. The vessel pressure correlates to a highest local electrode temperature. Liquid temperature measurement at the diaphragm provides a variable that is useful for estimation of the thermal ablation environment. These measurements are independent of the locations of the hot and cold areas on the electrode surface, which independence, among other benefits, is considered to overcome certain limitations considered to be responsible for temperature measurement errors in prior designs that are more dependent upon locality of thermal heating.

According to a further embodiment, an algorithm is provided that estimates the hottest temperature of the ablated tissue based upon at least one of, or combinations thereof, the vessel pressure, coolant fluid temperature, and the applied RF power. According to one further feature, the algorithm is provided in computer readable medium. According to another feature, a processor is provided that applies the algorithm in a manner useful in forming such estimation. In a further feature, the processor is coupled to the computer readable medium and accesses the algorithm from the computer readable medium for use in calculating the estimate. In still a further mode, a controller is also provided and is adapted to be coupled to the processor in a manner such that the estimation is used at least in part to control energy delivery at the thermal emitter, such as an electrode.

Another aspect of the invention is a tissue hyperthermia system that includes an energy emitter configured to be positioned at a location associated with a region of tissue of a body of a patient and that is actuatable at the location to an operating mode that emits energy into the region of tissue and heats to at least a threshold temperature. A two-phase energy transfer system is provided in the system and includes a material is thermally coupled to the energy emitter. The material undergoes a phase transformation between a first phase and a second phase when the energy emitter is heated to at least the threshold temperature.

Another aspect of the invention is a tissue hyperthermia system that includes a temperature monitoring system that is configured to estimate a regional temperature associated with an energy emitter that is actuated into an operational mode that emits energy into a region of tissue of a body of a patient and that heats to at least a threshold temperature. This estimate is based at least in part upon at least one parameter associated with a phase change between a first phase and a second phase of a material that is thermally coupled to the energy emitter.

Another aspect of the invention is a tissue hyperthermia system that includes a temperature controlled actuator configured to be coupled to an energy emitter assembly and to actuate the energy emitter into an operating mode that emits energy into a region of tissue of a body of a patient and that heats to at least a threshold temperature. The temperature controlled actuator is further configured to control the output of the energy emitter in the operating mode based at least in part upon an estimated regional temperature associated with the energy emitter at or above the threshold temperature. The estimated temperature used is based at least in part upon at least one monitored parameter associated with a phase change between a first phase and a second phase of a material that is thermally coupled to the energy emitter.

Another aspect of the invention is a tissue hyperthermia system that includes an algorithm stored in a computer readable medium. The algorithm is adapted to estimate a regional temperature associated with an energy emitter actuated to an operational mode that emits energy into a region of tissue of a body of a patient and that heats to at least a threshold temperature. The estimate is based at least in part upon at least one monitored parameter associated with a phase change between a first phase and a second phase of a material that is thermally coupled to the energy emitter.

Another aspect of the invention is a tissue hyperthermia system that includes an energy emitter that is configured to be positioned at a location associated with a region of tissue of a body of a patient. The energy emitter is actuatable at the location to an operating mode that emits energy into the region of tissue and that heats to at least a threshold temperature. An enclosed vessel thermally coupled to the energy emitter. At least one sensor is coupled to the enclosed vessel and configured to sense at least one parameter associated with the enclosed vessel. The at least one parameter is useful in estimating a regional temperature associated with the energy emitter. The at least one sensor is configured to be coupled to a monitoring system adapted to monitor the at least one sensed parameter for use in estimating the regional temperature.

Another aspect of the invention is a tissue hyperthermia system that includes a temperature monitoring system configured to estimate a regional temperature associated with an energy emitter that is actuated to an operational mode that emits energy into a region of tissue of a body of a patient and that heats to at least a threshold temperature. The estimate is based at least in part upon at least one sensed parameter associated with an enclosed vessel that is thermally coupled to the energy emitter.

Another aspect of the invention is a tissue hyperthermia system that includes a temperature controlled actuator configured to be coupled to an energy emitter and to actuate the energy emitter into an operating mode that emits energy into a region of tissue of a body of a patient and that heats to at least a threshold temperature. The temperature controlled actuator is configured to control the output of the energy emitter in the operating mode based at least in part upon an estimated regional temperature associated with the energy emitter at least at the threshold temperature. The estimated regional temperature used is based at least in part upon at least one monitored parameter associated with an enclosed vessel thermally coupled to the energy emitter.

Another aspect of the invention is a tissue hyperthermia system that includes an algorithm stored in a computer readable medium. The algorithm is adapted to estimate a regional temperature associated with an energy emitter actuated to an operational mode that emits energy into a region of tissue of a body of a patient and that heats to at least a threshold temperature. The estimate is based at least in part upon at least one monitored parameter associated with an enclosed vessel that is thermally coupled to the energy emitter.

Another aspect of the invention is a tissue hyperthermia system that includes an energy emitter configured to be positioned at a location associated with a region of tissue of a body of a patient, and that is actuatable at the location into an operating mode that emits energy into the region of tissue and that heats to at least a threshold temperature. Means for estimating a regional temperature associated with the energy emitter in the operating mode at the location are also provided.

Another aspect of the invention is a tissue hyperthermia system that includes an energy emitter configured to be positioned at a location associated with a region of tissue of a body of a patient, and that is actuatable at the location into an operating mode that emits energy into the region of tissue and that heats to at least a threshold temperature. Means for controlling an energy output of the energy emitter based at least in part upon an estimated regional temperature associated with the energy emitter in the operating mode at the location are also provided.

Another aspect of the invention is a tissue hyperthermia system that includes an algorithm stored in a computer readable medium. The algorithm is configured to estimate a regional temperature associated with an energy emitter assembly that is actuated to an operational mode that emits energy into a region of tissue of a body of a patient and that heats to at least a threshold temperature. The estimate is based at least in part upon a first monitored parameter associated with an energy output signal to the energy emitter assembly and a second monitored parameter associated with a sensed temperature associated with the energy emitter assembly.

According to one further mode of the system aspects that provide a two-phase energy transfer system with a transformational material thermally coupled to an energy emitter, the material is located within a substantially enclosed vessel coupled to the energy emitter. At least one sensor is coupled to the enclosed vessel and configured to sense at least one parameter associated with the enclosed vessel, or the material, or both. The at least one parameter varies in relation to the phase transformation of the material and is useful in estimating a regional temperature associated with the energy emitter, and the at least one sensor is configured to be coupled to a monitoring system adapted to monitor the at least one sensed parameter for use in estimating the regional temperature.

Aspects described hereunder that thermally couple a two-phase transformational material with a tissue energy emitter are also applicable in a similar manner as further contemplated aspects hereunder with respect instead to an enclosed vessel thermally coupled to the energy emitter. In one highly beneficial further mode, however, such an enclosed vessel is provided together with the material which is positioned within the enclosed vessel. Temperature estimation and/or output control to the emitter may be based upon one or more monitored parameters that relate to the material, one or more aspects of the vessel itself, or both.

In a highly beneficial further modes related to the two-phase heat transfer system aspects, or aspects providing an enclosed thermal vessel coupling to the emitter, or the combination thereof, an algorithm stored in a computer readable medium is configured to estimate a regional temperature associated with the energy emitter in the operational mode at the location based at least in part upon a first monitored parameter associated with an energy output signal to the energy emitter and a second monitored parameter associated with a sensed temperature associated with the material.

According to one particularly beneficial embodiment of modes herein described that provide an algorithm to estimate temperature based upon multiple parameters, and in particular using first and second parameters related to power output and a sensed temperature reading, respectively, the algorithm estimates the temperature based at least in part upon a simultaneous multivariable application of the first and second parameters.

According to one further feature of the system further to this embodiment, a processor is configured to be coupled to the computer readable medium and to access the algorithm and calculate the estimated regional temperature based upon the algorithm. According to another feature, an energy output controller is provided that is configured to control energy output to the energy emitter based upon the estimated regional temperature calculated by the processor. According to another feature, a temperature monitoring system is provided and is configured to monitor a temperature associated with the second parameter. In still another further embodiment, a power monitoring system that is configured to monitor a power signal associated with the first parameter used in the estimation.

According to another further embodiment considered still of particular benefit, the operating mode for the energy emitter comprises a modulated power operating mode of operation that comprises a modulated power signal over time. The temperature estimation algorithm is based at least in part upon a time dependent aspect of at least one of the first and second parameters with respect to the modulated power signal.

In a still further embodiment, the modulated power operating mode just described comprises a pulsed RF signal comprising a series of pulses with a pulse duration, latency period of separation between pulses, and cycle period that comprises a pulse duration plus latency period to a subsequent pulse, all over time. The temperature estimation algorithm is based at least in part upon a time dependent aspect of at least one of the first and second parameters with respect to the pulsed RF signal.

According to one further mode of aspects that utilize simultaneous multivariate monitored parameters of an energy emitter to estimate temperature, the algorithm comprises the relationship

Tₜ=Tₕ+[A*(T_{d})^{a}]/(P_{rf})^{b.}

According to this relationship, Tₜ represents an estimated maximum peak tissue temperature adjacent the energy emitter, Tₕ represents an estimated maximum temperature at the energy emitter, T_{d} represents an average monitored temperature associated with the energy emitter, P_{rf} represents power of RF energy delivered to the energy emitter, and A, a, and b are empirically derived constants.

It is to be appreciated according to this mode that the application of such multivariate algorithm may be employed for improved temperature monitoring and output control of existing standard RF ablation catheters, for example. Such combination is considered a further beneficial aspect herein contemplated. Moreover, further particular benefit is considered to result by further combination of this algorithm with the various other aspects herein contemplated.

For example, for aspects wherein a two-phase heat transfer system is provided thermally coupled to the energy emitter, a further mode uses an algorithm for estimating temperature based upon the following relationship:

Tₜ=Tₕ+[A*(T_{d})^{a}]/(P_{rf})^{b}.

According to this relationship, Tₜ represents an estimated maximum peak tissue temperature adjacent the energy emitter, Tₕ represents an estimated maximum temperature at the energy emitter, T_{d} represents an average monitored temperature of the material, P_{rf} represents power of RF energy delivered to the energy emitter, and A, a, and b are empirically derived constants.

For aspects wherein an enclosed vessel is thermally coupled to the energy emitter, the algorithm comprises the relationship:

Tₜ=Tₕ+[A*(T_{d})ₐ]/(P_{rf})^{b}.

In this setting, Tₜ represents an estimated maximum peak tissue temperature adjacent the energy emitter, Tₕ represents an estimated maximum temperature at the energy emitter, T_{d} represents an average monitored temperature of the vessel, P_{rf} represents power of RF energy delivered to the energy emitter, and A, a, and b are empirically derived constants. In addition, it is to be appreciated that where a two-phase heat transfer material is provided within such an enclosed vessel in thermal coupling with the emitter, the respectively monitored parameter of temperature may relate to the material directly, or another aspect of the vessel (eg. which may indirectly provide temperature of the material), or both, to suit the nature of the thermal coupling and sensing configuration employed.

According to still further highly beneficial modes of the present embodiments, the energy emitter provided in relation to other features described comprises an electrode. An electrical power generator coupled to the electrode actuates it to emit energy into tissue for therapy. In a particular embodiment, the electrical power generator comprises a radiofrequency (RF) power generator.

According to other modes, the energy emitter may be for example an ultrasound transducer, a microwave element, or a thermal conductor or other form of energy emitter wherein local heating of the emitter itself is useful to monitor and control in order to optimize therapeutic and safety results.

In still further modes, a delivery system is provided that is configured to deliver the energy emitter to the location which is within the patient's body. According to one highly beneficial embodiment, the delivery system comprises a delivery catheter with a proximal end portion and a distal end portion. The energy emitter and thermally coupled two-phase energy transfer system, and/or enclosed vessel, are located along the distal end portion. The distal end portion is adapted to be positioned at the location with the proximal end portion located externally of the location.

According to another further mode for the energy emitter where employed in thermal coupling with an enclosed vessel, the energy emitter comprises an annular shell that circumscribes an interior reservoir passageway extending between first and second substantially closed ends such that the reservoir passageway comprises a substantially enclosed vessel. A coolant material is located within the substantially enclosed vessel. In one further embodiment considered to provide substantial further benefit, the energy emitter comprises a sintered metal interior within an outer solid shell. The sintered interior may be for example sintered silver or platinum. Further more particular beneficial features considered to provide still further highly beneficial results are elsewhere herein described. In one such particular embodiment, the sintered metal comprises sufficient porosity to provide wicking of the coolant material into the pores.

According to yet another highly beneficial mode related to aspects employing a two-phase material heat transfer system, the phase change of the material is between a first phase that is a liquid phase, and a second phase that is a vapor phase. In one particular embodiment, the material is substantially in the first liquid phase at body temperature, such as according to one embodiment liquid water in the first phase and water vapor in the second phase. Various other further embodiments are herein described and considered of further benefit and value.

According to another mode related to aspects providing an enclosed vessel thermally coupled to the energy emitter, one end of the substantially enclosed vessel comprises a diaphragm, which may be for example in one highly beneficial further embodiment an adiabatic material. In a further embodiment, a pressure monitoring system is coupled to the vessel via the diaphragm. In one particular further feature, the pressure monitoring system may include a strain gauge coupled to the diaphragm.

According to another mode of the various aspects described and that is considered of particular benefit and value, the estimated temperature provided by the respective systems comprises an estimated hottest temperature along the energy emitter. In another also highly beneficial mode, the estimated temperature comprises an estimated maximum peak temperature in the region of tissue. Further beneficial embodiments are also herein provided with respect to particular threshold temperatures at which the respective systems and related methods provide particular benefit and use.

According to yet a further highly beneficial mode of the various aspects employing a two-phase heat transfer system thermally coupled to an energy emitter, the two-phase transformational material is adapted to actively cool the energy emitter via the phase transformation during the operating mode for the energy emitter.

Further aspects, modes, embodiments, and features contemplated under the present invention include the various methods related to the systems herein shown and described. Such novel methods are considered to provide particular benefit and value, and are considered further independent aspects contemplated hereunder, whether or not put to use in combination with the novel systems herein disclosed, or with other systems otherwise available in the art to the extent such methods are so applicable.

Each of the aspects, modes, embodiments, and features herein described is considered of independent benefit and value, without requiring their combination with the others. In addition, however, such combinations are also considered of still further particular benefit and use, and as such are considered further aspects of the present invention.

Still further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

For purposes of illustration, by way of example only, the application is directed primarily to endovascular ablation catheters of the type used for cardiac ablation. The invention will be more fully understood by reference to the following drawings which are for illustrative purposes only:

FIG. 1 shows a partially cross-sectioned view of an RF-ablation catheter electrode in combination with a two-phase change heat flow and transfer vessel.

FIG. 2 shows a schematically illustrated graph representing certain aspects related to exemplary thermodynamic operating conditions inside an electrode.

FIG. 3 shows a schematically illustrated graph representing an exemplary temperature environment of an ablation electrode.

FIG. 4 shows a block diagram of a temperature monitoring and control system.

FIG. 5 shows a schematically illustrated graph representing one pulsed RF mode of an RF modulation system.

Reference Numerals

10 - hollow electrode
11 - metal shell
12 - diaphragm
13 - capillary wick
14 - RE power connection
15 - vapor region
16 - pressure equalization channel
17 - coolant-filled vessel
18 - pressure transducer with output Sₕ
19 - temperature transducer with output S_{q}
20 - catheter shaft
21 - system console
22 - RF generator
23 - power meter
24 - processor
25 - closed-loop selector
26 - power controller
27 - display
subscripts:
b - refers to body temperature (37° C.)
d - refers to diaphragm temperature
h - refers to electrode hot spot
t - refers to tissue hot spot

### DETAILED DESCRIPTION OF THE INVENTION

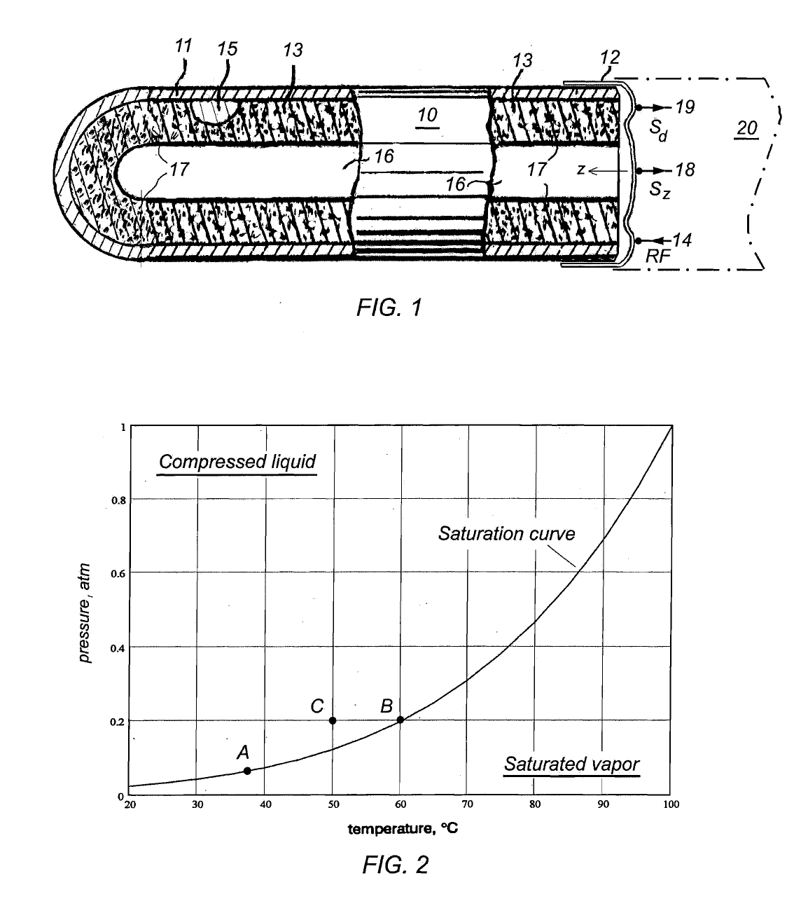

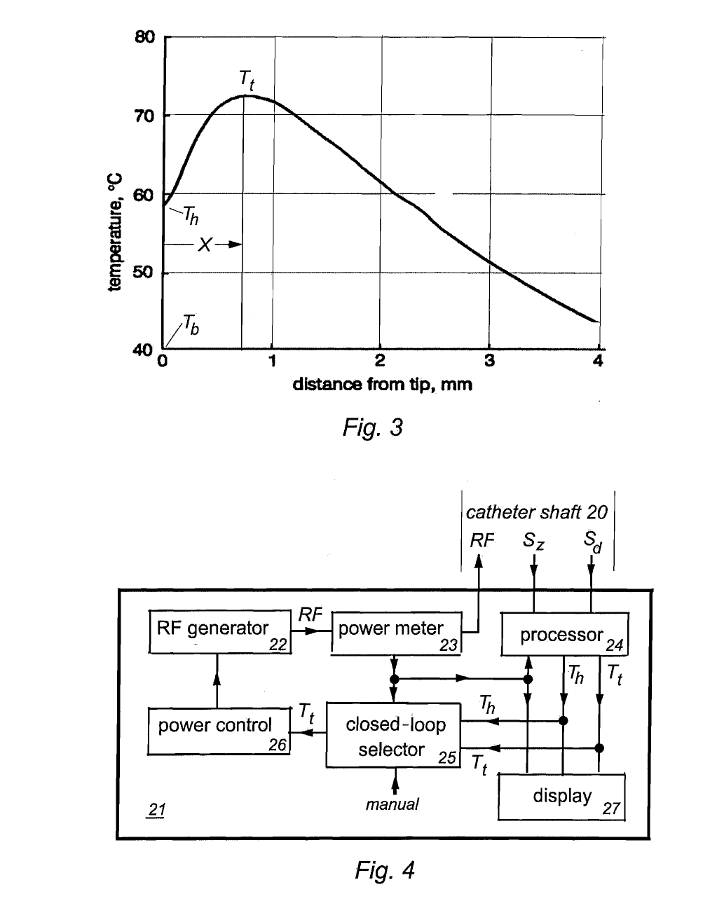

Referring more specifically to the drawings, for illustrative purposes the present invention is embodied in the apparatus generally shown in FIG. 1-5. It will be appreciated that the apparatus may vary as to configuration and as to details of the parts, and that the method may vary as to the specific steps and sequence, without departing from the basic concepts as disclosed herein.

Electrode Design

FIG. 1 shows the design of a hollow ablation electrode 10. Walls of electrode 10 are formed by a domed cylindrical metal shell 11. The following more detailed description of electrode 10 is provided as one exemplary embodiment in order to provide an illustrative example in significant detail in order to present a full and complete understanding of how the broad aspects of the present invention may be employed in at least one particular manner and device.

According to such illustrative example, the electrode 10 may comprise a hollow shell about 3 mm in diameter and about 8 mm long, and may be made of for example of platinum or silver (which may be for example a foil) with a wall thickness of about 0.2 mm. The interior of the shell may be manufactured for example using, powder metallurgy techniques; in such case the interior may be for example tightly filled with sintered silver particles in a micron diameter range.

According to the embodiment shown in FIG. 1, an axial blind hole, which may be for example 1 mm in diameter in the detailed illustrative embodiment shown and described, is drilled through the sintered interior. The blind hole serves as a pressure equalization channel 16 and the remaining sintered material serves as a capillary wick 13. In manufacture, the interior of shell 11 is filled with distilled and degassed water used as a coolant to substantially fill the vessel and saturate pores of the wick 13. A diaphragm-cap is also shown and described. The interior of shell 11 and diaphragm cap 12 forms a closed coolant-filled vessel 17.

In order to keep the liquid-vapor equilibrium pressure of the coolant liquid below the ambient atmospheric pressure at room temperature, the process of sealing the hollow electrode is done in partial vacuum. In this setting, vessel 17 is filled only with water, though the water may be in equilibrium with some residual vapor, which may occupy for example about 5% of the vessel volume.

Providing the wick feature in the electrode provides a particular further beneficial embodiment to enhance maintaining, and in many cases ensuring, the presence of water at an electrode hot spot to replace losses due to evaporation. Liquid may also be returned by capillary forces in wick 13 back to hotter areas of bubble formation so that the inner surface of the hollow electrode 10 generally remains wet. There are several materials and constructions considered suitable for wick 13 structure, including without limitation the following examples: screen, grooves, felt, and sintered powder. In particular, a sintered powder metal wick offers an advantage in catheter applications because it works in any orientation, even against gravity (i.e., the hot spot above the cold spot).

The porosity of the wick generally presents a compromise of inversely affected choices. The porosity thus may be chosen to meet a particular need to accommodate one or more related parameters, such as for example without limitation: electrode size, channel size, materials chosen, desired temperature, and coolant liquid. The porosity desirably provides high capillary pumping that is generally more directly proportional to pores being smaller, but a low flow-path resistance that is generally more directly proportional pores being larger. High wick permeability offers low fluid resistance and allows the wick to recharge as vaporization takes place. More liquid is supplied during the application of heat, and therefore, more heat can be transferred without the wick drying out. One way to accomplish the desired porosity is by controlling or choosing a particular size powder in the sintering process.

A sintered powder metal wick also has the ability to handle high heat fluxes. According to one particularly beneficial embodiment, the sintered powder wick may be for example about 50% porous and makes intimate contact with outer metal shell 11. Further to this embodiment, a large surface area is thus available for evaporation. Commercial sintered powder wicks handle for example about 50 W/cm². Scaling a wick of area illustrated in connection with FIG. 1 can effectively handle about 14 Watts of axial heat flow through the electrode. This is quite adequate for RF cardiac ablation applications, for example.

The electrode, including a sintered powder wick structure, can be bent or otherwise formed in different shapes, allowing more complex electrode geometries than shown in the illustrative embodiment here. A sintered silver wick is particularly beneficial because the heat exchange function is enhanced by the high thermal conductivity of silver. These attributes make the sintered silver powder wick 13 a highly beneficial structure contemplated according to the present embodiments. However, it is to be understood that other suitable sintered wick materials may be chosen, including others with even higher thermal conductivity, including for further example but without limitation diamond powder and carbon fibers.

Pressure in the vessel is measured by pressure transducer 19 located on diaphragm 12. Pressure measurement can be accomplished by a number of well-known techniques. One particular example measures diaphragm deflection in direction Z by diffused retro-reflection from the exterior surface of diaphragm 12 using fiber optics. Another suitable implementation of the pressure transducer is by a strain gage attached to diaphragm 12. A fiber-optic technique avoids electrical interference from the RF field. In any case, diaphragm deflection may be empirically correlated to pressure using experimental models for a particular set of design parameters chosen. A pressure indicating signal derived from the pressure transducer 18 is designated as S_{Z} and, as shown and described in further detail elsewhere hereunder, is more directly related to the electrode hot spot temperature than previously described monitoring systems and techniques. Temperature transducer 19 provides a signal S_{d} indicative of diaphragm temperature. Since the diaphragm is an adiabatic surface, its temperature represents the average liquid temperature of the vessel fluid.

While water is previously mentioned here in the illustrative embodiments as the coolant working fluid, other suitable fluids may be provided, such as for example but without limitation an alcohol (e.g. methanol) or R-113 refrigerant. Or, additives may be included in the base liquid, such as water, in order to modify (e.g. lower) the boiling temperature to the appropriate range intended in operation. For example, a combination of water and an alcohol, such as methanol, may be used which allows for a lower boiling point than simple water but higher biocompatibility than highly concentrated alcohol. Other additives such as salt (eg. NaCl) may be added to the water to also lower its boiling point.

According to certain particularly beneficial embodiments, the coolant fluid, in general, should have appropriately high latent heat of vaporization, appropriately high surface tension for effective capillary wicking flow, and appropriately low viscosity for little flow resistance. The coolant should also be relatively chemically inert and have relatively low toxicity as generally desired for the in-vivo medical applications herein contemplated in the preferred embodiments. However, as the material is intended to be contained within the associated vessel within the catheter, this may not be absolutely necessary for an appropriately confirmed robust containment within that vessel during intended modes of use. In a desired temperature-regulation region, it is desirable that the working fluid is present in both liquid and vapor phases and be at reasonable pressure for vessel integrity.

Operational Thermodynamics & Electrode Hot-Spot Temperature

Properties of water under varying temperature and pressure conditions are schematically illustrated in FIG. 2. The conditions on the saturation curve correspond to equilibrium between vapor and liquid. To the upper left of the saturation curve, water is in a compressed (subcooled) liquid state, where vapor bubbles are metastable and will spontaneously condense to liquid, in the process warming the liquid by giving up its latent heat of vaporization. To the lower right of the saturation curve, steam is in a superheated state and water droplets in this state will spontaneously evaporate, cooling the steam by absorption of the latent heat of vaporization.

Before RF power is applied to electrode 10, the coolant in vessel 17 is generally at body temperature, or typically about 37° C., which generally corresponds to an equilibrium pressure of about 0.07 atm for this particular coolant liquid. This represents thermodynamic state A, shown on the illustrative equilibrium curve in FIG. 2. As RF power is applied to electrode 10, an RF field generates heat in adjoining tissue and blood. A resulting increase in external surface temperature of electrode 10 is uneven because some areas absorb the heat flow created by the RF while others dissipate this heat into adjoining colder tissue and blood.

At some instant of time, the hottest spot is often concentrated at a particular region, illustrated in FIG. 1 as region 15. The external heat flux elevates the temperature at region 15, and the corresponding conditions are represented by illustrative point B on the illustrative saturation curve shown in FIG. 2. At this illustrative point B, the hot spot temperature Tₕ is about 60° C. and the corresponding pressure Prₕ is about 0.2 atm. The vapor bubbles at elevated pressure penetrate through the pores of the wet wick 13 into adjoining bulk liquid. Pressure inside the vessel 17 equalizes quickly through the equalization channel 16. The average water temperature is T_{d} (e.g., illustrated at 50° C.) so the liquid in container 17 is represented by point C and the liquid is in an overpressured state. Vapor bubbles created in region 15, upon entering into the liquid, quickly liquefy and dissipate their heat of vaporization. The liquid warms up, increasing the dissipation into adjoining blood and tissue through the chamber walls in contact with the electrode, until the dissipation is equal to the heat input.

In this system, there is a consistent relationship between Tₕ and Prₕ. In a closed vessel (eg. with limited available change of volume), there can be no net increase in the proportion of vapor which takes up much more volume than liquid. In this case the point of highest vessel temperature, Tₕ and pressure Prₕ are determined by the saturation curve. The temperature Tₕ can be readily determined from the pressure Prₕ and the equilibrium curve of the coolant. More specifically, this relationship between variables may be empirically established via simple experimental modeling for a particular set of chosen design and operating parameters, such that in the clinical operating environment employing those parameters the monitored information for Prₕ may be used to accurately estimate Tₕ. It is further noted that vessel pressure Prₕ and therefore temperature Tₕ are independent of the location of hottest and coldest spots on the surface of electrode 10.

During ablation, the temperature of the coolant fluid, T_{d}, increases until RF heating is equal to heat flowing out of the electrode into the cooler regions of adjacent tissue and blood. T_{d} then equilibrates throughout the vessel and specifically at the adiabatic surface 18 of the diaphragm. Temperature and displacement sensors placed on the diaphragm provide reliable, location-independent measurement of the overall electrode environment.

It is further noted that, in relation to a container filled with gas only, pressure times volume is generally directly proportional to temperature according to the following “Ideal Gas Law” equation: PV=nRT. An “ideal gas” according to this equation is one whose physical behavior is accurately described by the ideal-gas equation. Here, the constant R is called the gas constant, and the following general standards of terminology typically apply. The value and units of R depend on the units used in determining P, V, n and T. Temperature, T, according to this overall equation is expressed on an absolute-temperature scale (K). The quantity of gas, n, is normally expressed in moles. The units chosen for pressure and volume are typically atmospheres (atm) and liters (l), however, other units may be chosen.

Thus, according to the foregoing, in further embodiments herein contemplated, the material contained within the enclosed pressure vessel that is thermally coupled to an energy emitter may be an ideal gas. In this setting, pressure of the vessel is directly proportional to (and thus useful as a very predictable predictor of) temperature in the vessel according to well settled physics of the Ideal Gas Law.

However, notwithstanding certain benefits provided via this further gas-filled vessel embodiment, however, complete containment of gases is very difficult to achieve. Moreover, the phase-change embodiments elsewhere herein described afford certain particular benefits that are unique to those embodiments and highly desirable in many circumstances.

Although there are certain similarities between the processes described above and other systems generally referred to as “heat pipes,” there are also certain differences incorporated into various embodiments herein shown and described in varying degrees of detail. As the term “pipe” implies, such a device has a distinct heat-absorbing evaporation region and a heat-releasing condensation region where vapor condenses against the vessel wall, with the two regions separated by a length of pipe. While further contemplated embodiments herein contemplated provide a catheter implementation of a heat pipe where the evaporator is the electrode connected by a thin flexible pipe through a catheter shaft to a more proximally placed heat sink, the small diameter and flexibility requirements for such a heat pipe can render this approach impractical in certain circumstances.

Accordingly, in the other present embodiments featured hereunder, unlike in the heat pipe, the vapor condensation takes place in sub-cooled liquid and not at the condenser walls. Vapor is absorbed into adjacent liquid with the net result of a heating of the liquid. A highly beneficial feature is thus provided via a particular relationship between the vessel pressure and the vessel hot spot temperature. Pressure measurement in effect provides a hot-spot temperature measurement regardless of its location in the changeable distribution of the wall temperature. Such present embodiments are thus optimized for accurate measurement of hottest electrode temperature more particularly than to maximize heat flow.

Algorithm for Tissue Temperature Estimation

The invention provides for at least two measurements that can be made continuously during RF ablation and which can be used to control RF power output—the temperature of the coolant inside the electrode, measured at the diaphragm (Sd) and the pressure inside the vessel (Sz). The purpose of the algorithm is to accurately estimate the maximal temperature of the tissue adjacent to the electrode in real time using these variables. Since peak tissue temperature may be found within the tissue rather than at the site of contact, simply limiting the maximal electrode temperature (Th) may not be sufficient to prevent excessive heating.

The gradient of tissue temperature between the hottest spot on the electrode (Th) and the hottest spot within the tissue (Tt) is a function of heat flow in this region. The difference between Tt and Th increases with increasing dissipation of heat from the ablating electrode and electrode-tissue interface. The magnitude of heat dissipation from the ablation site is reflected by the efficiency of heating (the ratio average electrode temperature Td and applied RF power Prf. Thus an algorithm for estimation of maximal tissue temperature is given by:

Tₜ=Tₕ+[A*(T_{d})^{a}]/(P_{rf})^{b} (1)

Here A, a, and b are calibration constants. These may be determined for example to suit a particular chosen implementation of detailed design and operating parameters based upon experimental modeling. For example, one of ordinary skill based upon a full a detailed review of this disclosure may establish a best fit to a wide range of conditions based on computer modeling and in-vitro testing, and which may be further verified in animal studies where operating parameters and detailed measurements may be taken to arrive at the empirical results applicable to future clinical uses.

In general, this invention provides three readily available variables: pressure and temperature at the diaphragm and the RF power reading. They can be used in a variety of algorithms for highest tissue temperature and other useful ablation parameters such as RF tissue dosimetry.

A block diagram of a control implementation is shown in FIG. 4. Three input signals are presented to the processor: A vessel pressure signal S_{z} and a diaphragm temperature signal S_{d} are transmitted on the catheter shaft. The RF power reading signal P_{rf} is generated in the power meter 23. Based on saturation data for the vessel coolant, the processor converts input signal S_{z} to output signal Tₕ representing the electrode hot spot. Based on the algorithms above, the processor estimates the highest tissue temperature Tₜ. The closed-loop selector 25 determines the variable that controls the RF ablation power. The default control variable is Tₜ, but other variables or open-loop operation can be selected. The controlled RF power is transmitted through the catheter shaft to the ablation electrode.

While the highly beneficial embodiment of multivariable control is based on variables partially derived from a hollow electrode filled with liquid and vapor, other embodiments include multivariable algorithms based on variables otherwise derived, e.g., based on temperature and RF power indication in present day catheters.

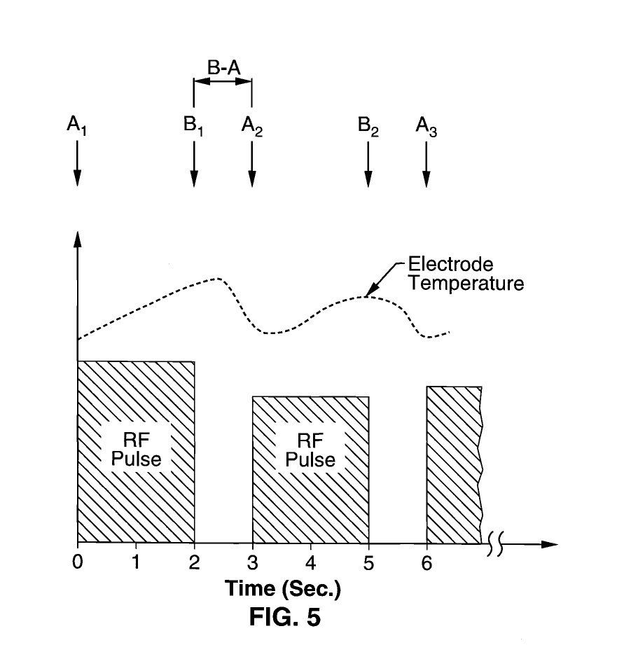

Another algorithm implementation is shown in FIG. 5. The heating RF pulses are turned on and off. RF pulses at varying amplitude start at points A and end two seconds later at points B. Power is turned off during interval BA lasting 1 sec. used to calculate tissue temperature Such temperature calculation determine the amplitude of the next AB pulse so as to maintain the desired average tissue temperature dosimetry. If the heating RF power is turned off the heat flow towards the electrode will continue for some time and the temperature of the electrode will increase if the tissue temperature is higher than the electrode temperature, as shown by the dashed electrode temperature function. The electrode temperature time function, right after RF power it turned off, contains therefore important information on the tissue thermal condition that cannot be obtained from electrode temperature alone. The essential aspect of the estimation algorithm is that the RF power is modulated and the resulting time function or functions of the electrode temperature are analyzed to obtain the estimate of highest temperature in the remote tissue location.

The minimum requirement for above algorithm is the availability of the power level and at least one electrode temperature signal. Such variables are available on virtually all present day ablation instruments that can therefore be combined with the use the above algorithm in an overall system as improved according to the applicable beneficial embodiments of the present invention. Further improvement in accuracy can be obtained using a two-phase electrode system, as illustrated schematically in FIG. 4, that provides three input variables RF, Sz, and Sd. Multivariable systems can be also used using other type of sensors.

The present disclosure has described what are believed to be accurate representations of various physical mechanisms and relationships between parameters within a thermodynamic system associated with ablation electrodes. However, it is to be appreciated that the various broad aspects of the invention should not be so limited or bound by theory except where expressly stated so, and in particular reference to the claims below.

For example, one such broad aspect provides a material thermally coupled to an electrode (or other heating element) and that undergoes a phase transformation upon the electrode reaching at least a certain threshold temperature. Another broad and independent aspect, though in certain further modes associated in combination with the previous aspect just described, uses a pressure measurement within an enclosed vessel associated with an electrode to estimate temperature of the electrode. A further mode of this aspect, though also considered independently beneficial, further includes combining a temperature reading together with the pressure reading for more accurate estimation of the target electrode and/or tissue temperature. Each of these novel aspects is considered independently beneficial, and without further limitation, regardless of the particular specific formulaic representations or relationships between parameters that actually exist within the related elements or combination system.

Notwithstanding the foregoing, however, it nevertheless remains that these physical characteristics and mechanisms of features, and relationships between related features, as herein described are still considered of further particular benefit and value, and thus constitute further independent aspects to the extent herein described.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A tissue hyperthermia system, comprising:
an energy emitter;
wherein the energy emitter is configured to be positioned at a location associated with a region of tissue of a body of a patient;
wherein the energy emitter is actuatable at the location to an operating mode that emits energy into the region of tissue and heats to at least a threshold temperature;
a two-phase energy transfer system comprising a material that is thermally coupled to the energy emitter; and
wherein the material undergoes a phase transformation between a first phase and a second phase when the energy emitter is heated to at least the threshold temperature.

2-8. (canceled)

9. A tissue hyperthermia system, comprising:
an energy emitter;
wherein the energy emitter is configured to be positioned at a location associated with a region of tissue of a body of a patient;
wherein the energy emitter at the location is actuatable at the location into an operating mode that emits energy into the region of tissue and that heats to at least a threshold temperature; and
means for estimating a regional temperature associated with the energy emitter in the operating mode at the location.

10. A tissue hyperthermia system, comprising:
an energy emitter;
wherein the energy emitter is configured to be positioned at a location associated with a region of tissue of a body of a patient;
wherein the energy emitter at the location is actuatable at the location into an operating mode that emits energy into the region of tissue and that heats to at least a threshold temperature; and
means for controlling an energy output of the energy emitter based at least in part upon an estimated regional temperature associated with the energy emitter in the operating mode at the location.

11-37. (canceled)

38. The method of claim 67, wherein:
the energy emitter comprises an ultrasound transducer; and
the actuating comprises actuating the ultrasound transducer to an operating mode that emits ultrasound energy into the region of tissue.

39. The method of claim 67, wherein:
the energy emitter comprises a microwave element; and
the actuating comprises actuating the microwave element to an operating mode that inductively heats the region of tissue.

40. The method of claim 67, wherein:
the energy emitter comprises a thermal conductor; and
the actuating comprises actuating the thermal conductor to the operating mode that heats the thermal conductor to conductively heat the region of tissue.

41-66. (canceled)

67. A tissue hyperthermia method, comprising:
positioning an energy emitter at a location associated with a region of tissue of a body of a patient;
actuating the energy emitter at the location to an operating mode that emits energy into the region of tissue and that heats to at least a threshold temperature;
thermally coupling a material of a two-phase energy transfer system to the energy emitter; and
wherein the material undergoes a phase transformation between a first phase and a second phase when the energy emitter is heated to at least the threshold temperature.

68-74. (canceled)

75. A tissue hyperthermia method, comprising:
positioning an energy emitter at a location associated with a region of tissue of a body of a patient;
actuating the energy emitter at the location into an operating mode that emits energy into the region of tissue and that heats to at least a threshold temperature; and
a step for estimating a regional temperature associated with the energy emitter in the operating mode at the location and for controlling energy output to the energy emitter.

76-77. (canceled)

78. The method of claim 67, further comprising:
coupling a temperature monitoring system to the two-phase heat transfer system;
using the temperature monitoring system, estimating a regional temperature associated with the energy emitter that is heated at least to the threshold temperature; and
wherein the estimating is based at least in part upon at least one parameter associated with the phase transformation of the material.

79. The method of claim 67, further comprising:
coupling a temperature controlled actuator to the energy emitter;
using the temperature controlled actuator to actuate the energy emitter into the operating mode at the location;
using the temperature controlled actuator, controlling an output signal to the energy emitter in the operating mode based at least in part upon an estimated regional temperature associated with the energy emitter at or above the threshold temperature; and
wherein the estimated regional temperature used in the controlling is based at least in part upon at least one monitored parameter associated with the phase transformation of the material.

80. The method of claim 67, further comprising:
storing an algorithm on a computer readable medium;
using the stored algorithm, estimating a regional temperature associated with the energy emitter in the operating mode at the location; and
wherein the estimating is based at least in part upon at least one monitored parameter associated with the phase transformation of the material.

81. The method of claim 67, further comprising:
coupling a substantially enclosed vessel to the energy emitter;
positioning the material within the enclosed vessel;
coupling at least one sensor to the enclosed vessel;
using the at least one sensor, sensing at least one parameter associated with the enclosed vessel;
wherein the at least one sensed parameter varies in relation to the phase transformation of the material; and
using the at least one sensed parameter, estimating a regional temperature associated with the energy emitter.

82-83. (canceled)

84. The method of claim 67, further comprising:
storing an algorithm in a computer readable medium;
monitoring a first parameter associated with an energy output signal to the energy emitter;
monitoring a second parameter associated with a sensed temperature associated with the material;
using the algorithm, estimating a regional temperature associated with the energy emitter in the operational mode at the location; and
wherein the estimating is based at least in part upon the first monitored parameter in conjunction with the second monitored parameter.

85-90. (canceled)

91. The method of claim 84, wherein:
using the algorithm for estimating the regional temperature comprises using a simultaneous multivariable application of the first and second parameters.

92. The method of claim 91, further comprising:
coupling a processor to the computer readable medium;
accessing the algorithm with the processor; and
the estimating comprises using the processor to run the algorithm.

93. The method of claim 92, further comprising:
using an energy output controller to control energy output to the energy emitter based upon the estimated regional temperature calculated by the processor.

94. The method of claim 91, further comprising:
using a temperature monitoring system to monitor the sensed temperature associated with the second parameter.

95. The method of claim 91, further comprising:
using a power monitoring system to monitor a power signal associated with the first parameter.

96. The method of claim 91, wherein:
actuating the energy emitter into the operating mode comprises supplying the energy emitter with a modulated power signal that is modulated over time; and
the temperature estimation algorithm used in estimating the regional temperature is based at least in part upon a time dependent aspect of at least one of the first and second parameters with respect to the modulated power signal.

97. The method of claim 96, wherein:
the modulated power signal delivered to the energy emitter comprises a pulsed RF signal that comprises a series of pulses with a pulse duration, latency period of separation between pulses, and cycle period that comprises a pulse duration plus latency period to a subsequent pulse, all over time; and
the temperature estimation algorithm used to estimate the regional temperature is based at least in part upon a time dependent aspect of at least one of the first and second parameters with respect to the pulsed RF signal.

98. (canceled)

99. The method of claim 84, wherein:
the algorithm used to estimate the regional temperature comprises the relationship Tₜ=Tₕ+[A*(T_{d})^{a}]/(P_{rf})^{b}; and
Tₜ represents an estimated maximum peak tissue temperature adjacent the energy emitter, Tₕ represents an estimated maximum temperature at the energy emitter, T_{d} represents an average monitored temperature of the material, P_{rf} represents power of RF energy delivered to the energy emitter, and A, a, and b are empirically derived constants.

100. (canceled)

101. The method of claim 67, wherein the energy emitter comprises an electrode, and further comprising:
coupling an electrical power generator to the electrode; and
using the electrical power generator, actuating the electrode to the operating condition at the location.

102. The method of claim 101, further comprising:
using the electrical power generator, delivering a radiofrequency (RF) power signal to the electrode for actuating the electrode to the operating condition.

103. The method of claim 67, further comprising:
delivering the energy emitter to the location that is within the patient's body with a delivery system.

104. The method of claim 103, wherein:
the delivery system comprises a delivery catheter with a proximal end portion and a distal end portion;
the energy emitter is located along the distal end portion;
the two-phase energy transfer system is located along the distal end portion; and
positioning the distal end portion at the location with the proximal end portion located externally of the location.

105. The method of claim 67, further comprising:
providing the energy emitter as an electrode assembly that comprises an annular shell that circumscribes an interior reservoir passageway extending between first and second substantially closed ends such that the reservoir passageway comprises a substantially enclosed vessel; and
positioning a coolant material within the substantially enclosed vessel.

106. The method of claim 105, wherein:
the energy emitter comprises a sintered metal interior within an outer solid shell; and
the sintered metal comprises sufficient porosity to allow wicking of the coolant material into the pores.

107. The method of claim 67, wherein:
the first phase comprises a liquid phase; and
the second phase comprises a vapor phase.

108. The method of 84 claim 107, wherein:
the material is substantially in the first liquid phase at body temperature.

109. The method of claim 108, wherein:
the material comprises liquid water in the first phase and water vapor in the second phase.

110. The method of claim 67, wherein:
the material is characterized as having a boiling point at a threshold temperature that is less than about 100 deg C.

111-112. (canceled)

113. The method of claim 105, wherein:
one end of the substantially enclosed vessel comprises a diaphragm.

114. The method of claim 113, wherein:
the diaphragm comprises a substantially adiabatic material.

115. The method of claim 113, further comprising:
coupling a pressure monitoring system to the vessel via the diaphragm.

116. The method of claim 115, wherein:
the coupling of the pressure monitoring system comprises coupling a strain gauge to the diaphragm.

117. The method of claim 67, wherein:
the estimating comprises estimating a hottest temperature along the energy emitter.

118. The method of claim 67, wherein:
the estimating comprises estimating an estimated maximum peak temperature in the region of tissue.

119. The method of claim 67, wherein:
the threshold temperature is at least about 45 degrees C.

120. (canceled)

121. (canceled)

121. The method of claim 105, wherein:
the electrode comprises an end-electrode on an end-electrode ablation catheter.

122. The method of claim 121, wherein the end-electrode ablation catheter is deflectable, and further comprising deflecting the deflectable member while delivering the electrode to the location.

123. The method of claim 67, further comprising:
mapping a cardiac conduction signal in a heart of the patient and in order to identify the region of tissue to be treated.

124. The method of claim 67, further comprising:
cooling the energy emitter at least in part with the material via the phase transformation during the operating mode for the energy emitter.
